(19) United States
(12) Patent Application Publication
(10) Pub. No.: US 2004/0148023 A1
(43) Pub. Date: Jul. 29, 2004

(54) High gain wide range accommodating intraocular lens for implant into the capsular bag

(76) Inventor: Stephen K. Shu, Laguna Niguel, CA (US)

Correspondence Address: LEONARD TACHNER, A PROFESSIONAL LAW, CORPORATION, 17961 SKY PARK CIRCLE, SUITE 38-E, IRVINE, CA, 92614

(21) Appl. No.: 10/758,830

(22) Filed: Jan. 15, 2004

Related U.S. Application Data
(63) Continuation-in-part of application No. 09/788,041, filed on Feb. 15, 2001, now abandoned.

(51) Int. Cl. A61F 2/16

(52) U.S. Cl. 623/6.34; 623/6.37

(57) ABSTRACT

A high gain lens system for implant into the capsular bag after removal of the natural crystalline lens. A preferred embodiment of the invention comprises a combination of a positive or convex lens and a negative or concave lens. These two lenses are spaced from one another and their relative spacing and respective focal lengths determine their combined focal length. When the lens system is inserted into the capsular bag, two opposed haptic flanges on each side, extend toward the inner radial edge of the bag adjacent the ciliary muscles. When the muscles contract, the bag is stretched thereby compressing the haptic flanges together or at least toward one another. This action cause the two lenses to separate further from each other and the increased spacing between the positive and negative lenses shortens the focal length to permit focusing of objects at near distances. On the other hand, when the muscles relax, the bag relaxes also, the haptic flanges separate and the lenses come closer together. The reduced spacing between the positive and negative lenses, increases the focal length to permit focusing of objects at far distances.

### CROSS-RELATED APPLICATIONS

This applications is a continuation-in-part of U.S. patent application Ser. No. 09/788,041 filed Feb. 15, 2001 and now issued U.S. Pat. No. ______ from which priority is claimed.

### BACKGROUND OF THE INVENTION

1. Field of the Invention

The present invention relates generally to intraocular lens implants and more specifically to a high gain implantable intraocular lens which utilizes a dual lens device to reduce capsular bag action to achieve focal point accommodation with minimal stress on ciliary muscles.

2. Background Art

The most relevant prior art appears to be disclosed in U.S. Pat. No. 5,674,282 issued on Oct. 7, 1997 to Cumming. This disclosure also relates to an accommodating intraocular lens designed to be implanted in the human eye capsular bag after the natural crystalline lens has been surgically removed such as during cataract surgery. Accommodation is the muscle-controlled change in focal point of the eye lens such as for viewing an object near the eye after initially viewing a distant object.

The lens disclosed in the Cumming patent is a relatively simple convex lens and accommodation requires movement of the lens toward and away from the retina of the eye. Unfortunately, because the gain of a simple convex lens is low, the distance the lens must be actually moved within the eye to achieve a large focal length change, is relatively large. Requiring a large distance for lens movement means that achieving accommodation for near vision is difficult at best and may ultimately become impossible when the ciliary muscles become too weak or too tired to achieve the necessary lens movement.

It would be highly advantageous if an implantable intraocular lens for ciliary muscle accommodation control, could be designed to permit large focal point variation and range of accommodation with less muscle strain. In other words, a higher gain controllable lens, which provides needed accommodation with less movement and less muscle exertion, would be an extremely desirable improvement. One prior art attempt at an implantable complex lens device to provide muscle control accommodation is disclosed in U.S. Pat. No. 5,275,623 to Sarforazi wherein two spaced convex lenses have their spacing altered by muscle control of the capsular bag. However, a dual convex lens system still requires a substantial movement to accommodate large focal point variation.

### SUMMARY OF THE INVENTION

The present invention comprises a high gain lens system for implant into the capsular bag after removal of the natural crystalline lens, such as a result of cataract surgery. While the invention herein has certain characteristics in common with the prior art such as the aforementioned patents to Cumming and Sarforazi, its uniqueness resides in the achievement of a higher optical gain and greater range of accommodation which provides full focal length accommodation with less muscle exertion. A preferred embodiment of the invention comprises a combination of a positive or convex lens and a negative or concave lens. These two lenses are spaced from one another and their relative spacing and respective focal lengths determine their combined focal length. The two lenses are joined along their perimeters through a common, interface which may be either integral to or attached to the respective lenses. In the preferred embodiment, the interface is formed as a narrow, flexible joint which acts an annular pivot for controlling the spacing between the lenses. The interface extends radially from this joint along two substantially parallel planes around opposing portions of the lens structure forming a scissor-like pair of closely spaced haptic flanges on each of two opposed sides of the lenses.

When the lens system of the preferred embodiment is inserted into the capsular bag, the two opposed haptic flanges on each side, extend toward the inner radial edge of the bag adjacent the ciliary muscles. When the muscles contract, the bag is stretched thereby compressing the haptic flanges together or at least toward one another. This action cause the two lenses to separate further from each other and the increased spacing between the positive and negative lenses shortens the focal length to permit focusing of objects at near distances. On the other hand, when the muscles relax, the bag relaxes also, the haptic flanges separate and the lenses come closer together. The reduced spacing between the positive and negative lenses, increases the focal length to permit focusing of objects at far distances. Thus, the preferred embodiment permits muscle control of the focal length of the lens system. Moreover, it will be seen hereinafter that the amount of movement of the lenses relative to one another to achieve a given change in focal length is relatively small because the optical gain is high. Therefore, the invention herein constitutes a significant advance over the prior art by providing a practical lens system that permits easier and greater extent of accommodation using ciliary muscles attached to the capsular bag. A second embodiment of the invention employs the same two lenses of the first embodiment, but with a different spacing control implementation based on the same pivoting scissor technique. A third embodiment employs an enclosed ring forming a unitary pressure chamber where increased pressure separates the two lenses.

An important benefit of the lens system of all of the disclosed embodiments is the large range of focal lengths that result. Such a large range assures compensation for changing lens characteristics due to aging or infirmities such as nearsightedness.

### OBJECTS OF THE INVENTION

It is therefore a principal object of the present invention to provide an implantable intraocular high gain lens having a large range adjustable focal length which can be controlled by eye muscles.

It is another object of the present invention to provide a focal length adjustable implantable intraocular lens which has a higher gain and larger range than such lenses of the prior art.

It is still another object of the invention to provide a complex adjustable lens system for implanting into the capsular bag of the human eye, the lens system having a positive lens and a negative lens and yielding a high optical gain based upon the relative spacing of the two lenses.

It is yet another object of the invention to provide a high gain, large range, accommodating implantable lens system configured for aligning a positive lens and a negative lens as they are moved axially relative to one another to alter their combined focal point.

It is yet another object of the invention to provide an implantable, adjustable lens system the focal length of which may be varied over a sufficiently large range to accommodate near and far vision requirements even with changing characteristics over the entire adult life of a user.

It is yet another object of the invention to provide an implantable adjustable lens system wherein a muscle-activated scissor-like operation assures continuous alignment of the lenses of the system regardless of the application of unbalanced forces.

### BRIEF DESCRIPTION OF THE DRAWINGS

The aforementioned objects and advantages of the present invention, as well as additional objects and advantages thereof, will be more fully understood hereinafter as a result of a detailed description of a preferred embodiment when taken in conjunction with the following drawings in which:

FIG. 1 is a cross-sectional view of a human eye shown with the natural crystalline lens removed and replaced with a preferred embodiment of the invention;

FIG. 2 is a cross-sectional view of the preferred embodiment shown in a near vision configuration;

FIG. 3 is a cross-sectional view of the preferred embodiment shown in a far vision configuration;

FIG. 4 is a top view of the preferred embodiment;

FIGS. 5a and 5b are side views of alternative embodiments shown in relaxed and extended positions;

FIG. 5c is a three-dimensional view of the embodiment of FIGS. 5a and 5b;

FIG. 5d is a sectional view taken along lines A-A in FIG. 5c;

FIG. 6 is a top view of the alternative embodiment; and

FIG. 7, comprising FIGS. 7a and 7b, is a side view of another alternative embodiment shown in relaxed and extended positions;

FIG. 8 is a top view of the embodiment of FIG. 7; and

FIG. 9 is a ray diagram of the lens system of the invention.

### DETAILED DESCRIPTION OF A PREFERRED EMBODIMENT

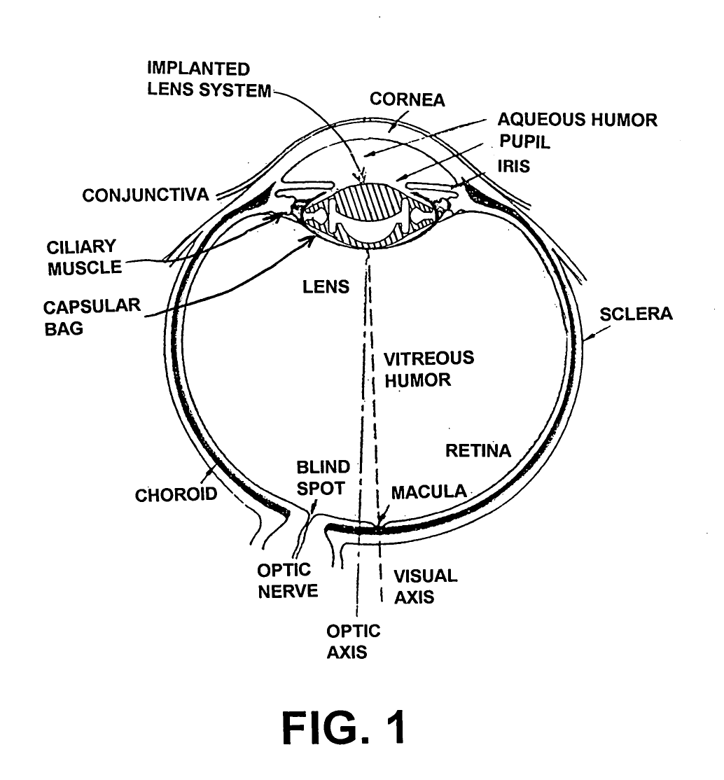

Referring to the accompanying figures, and initially FIG. 1, it will be seen that in a human eye immediately behind the cornea, the aqueous humor and the pupil, there is a capsular bag attached to ciliary muscles. In a normal human eye, the capsular bag is filled with a crystalline lens which the bag fully encloses. However, in FIG. 1, it is assumed that the eye has undergone surgery to cut through a portion of the capsular bag and to remove the entire crystalline lens. A technical description of this type of eye surgery, which is commonly done to remedy the condition know as “cataracts”, is provided in the aforementioned Cumming U.S. Pat. No. 5,674,282 which patent disclosure is incorporated herein by reference. It is further assumed that a lens system according to a preferred embodiment of the invention, has been installed into the capsular bag after the natural lens has been removed. It is further assumed that a portion of the capsular bag facing the cornea and immediately behind the iris, may have been permanently removed as part of the surgical process to remove the natural crystalline lens. However, the operation of the inventive lens system will not be significantly affected by such removal of a portion of the capsular bag as long as that portion of the bag to which the ciliary muscles are attached, remains intact.

It will be seen in FIG. 1, that the inventive lens system substantially fills the capsular bag extending radially to the inner radial surface of the bag and extending axially virtually to the anterior and posterior limits of the bag's inner surface.

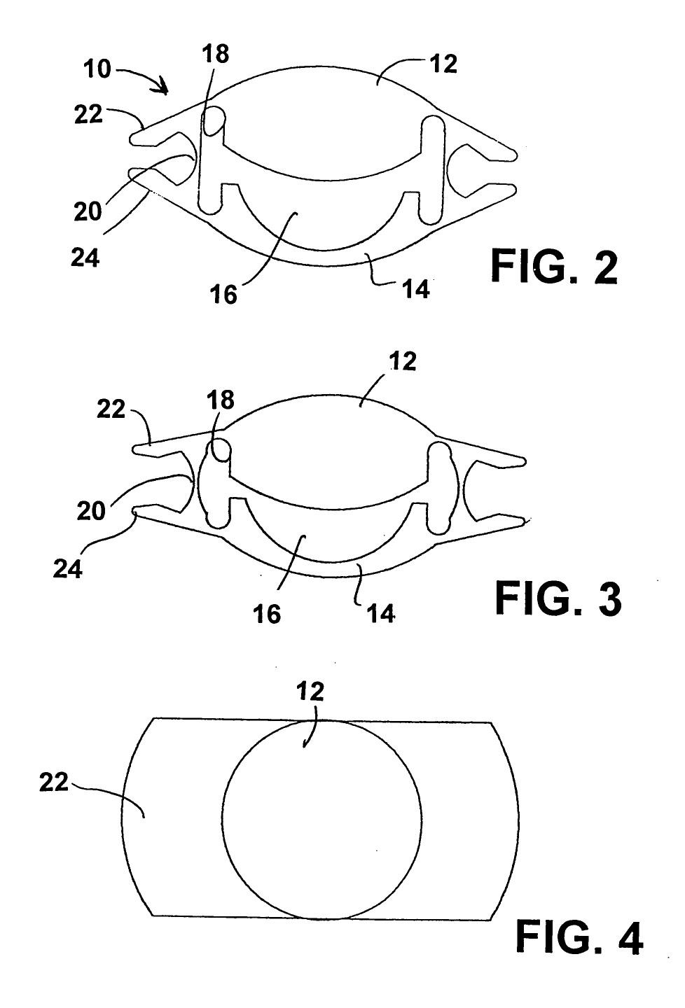

Referring now to FIGS. 2-4, it will be seen that a lens system 10 comprises a positive or convex lens 12 and a negative or concave lens 14. The lenses 12 and 14 are separated by a chamber 16 which terminates in an annular gap 18. The two lenses are connected along their radial edge at fulcrum 20 which radially separates lenses 12 and 14 from a pair of haptics 22 and 24 on each of the said lens system 10, the haptics providing a lever effect on the lenses much like a pair of scissors or pliers.

In operation, haptics 22 and 24 are either compressed together or relaxed apart. The lenses 12 and 14 do just the opposite of the haptics. Specifically, when the haptics 22 and 24 are compressed together by the ciliary muscles and capsular bag, the lenses separate, thereby increasing the width of chamber 16 to the extent shown in FIG. 2. In this configuration, the combined focal length of the two lenses 12 and 14, is shortened which is required for near distance viewing. On the other hand, when the haptics 22 and 24 are relaxed apart by relaxation of the ciliary muscles and capsular bag, the lenses come closer together, thereby decreasing the width of chamber 16 to the extent shown in FIG. 3. In this configuration, the combined focal length of the two lenses 12 and 14, is lengthened which is required for far distance viewing.

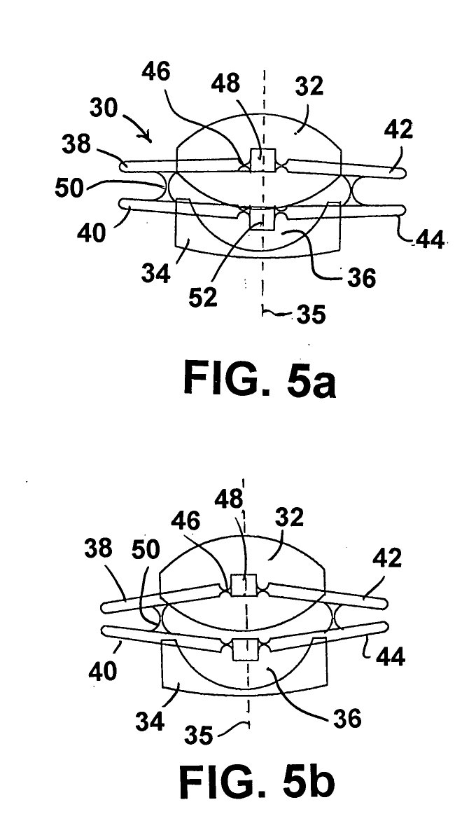

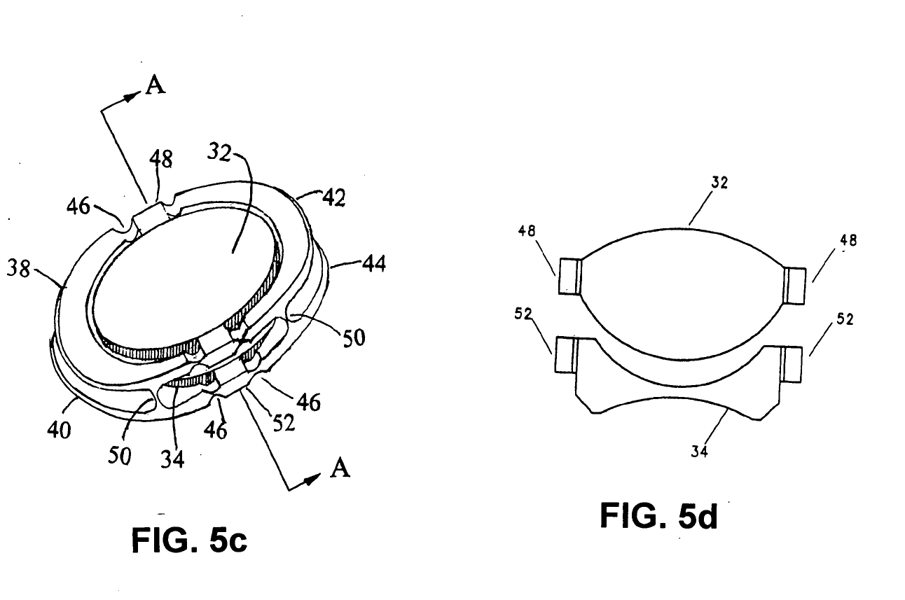

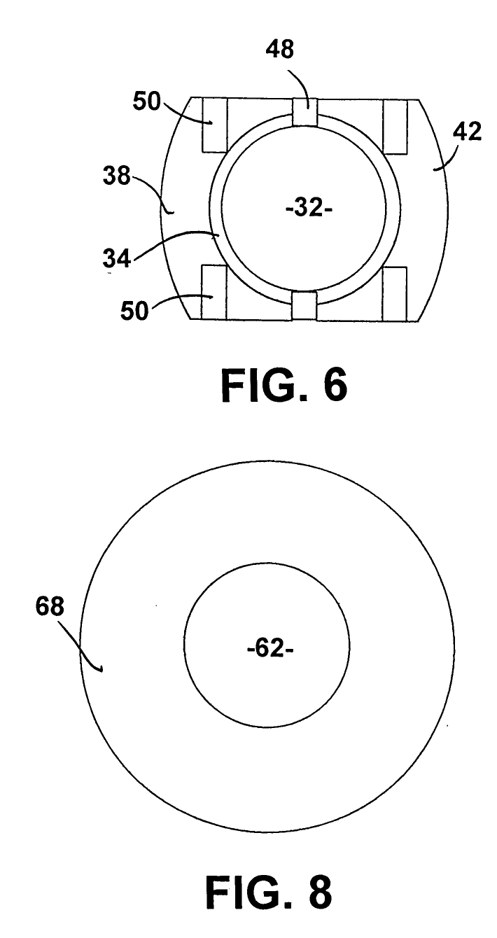

A second embodiment of the invention illustrated in FIGS. 5 and 6, operates in essentially the same way as the first embodiment of FIGS. 2 through 4. The principal difference resides in the mechanism to control the distance between the lenses. Specifically, a lens system 30 comprises a positive lens 32 and a negative lens 34 separated by a chamber 36. The spacing between the lenses 32 and 34 is controlled by haptics 38, 40, 42 and 44. Each such haptic is attached to a lens by means of a flex interface (i.e., interface 48 for lens 32 and interface 52 for lens 34). Each flex interface provides a bend point such as point 46. A fulcrum 50 on each side of lenses 32 and 34 provides a pivot for compression of haptics 38, 40, 42 and 44 which increases the distance between lenses 32 and 34 analogous to FIG. 2 for the first embodiment, or permits a relaxation of the haptics, which decreases the distance between the lenses analogous to FIG. 3 of the first embodiment.

As shown best in FIGS. 5c and 5d, as haptics 38 and 40 move toward each other and as haptics 42 and 44 move toward each other, lenses 32 and 34 separate from one another, but remain co-axial at all times even if muscular pressure on opposing pairs of haptics is unequal. Hinges 46 and flex interfaces 48 and 52 may be preferably made of a plastic and forces on them can be preset either positively or negatively and can be adjusted even after implanted using heat or laser light. By employing relatively wide interfaces 48 and 52, twisting of the lenses out of co-axial alignment is prevented to assure good optical characteristics over many years of operation. The distances between the closest surfaces of lenses 32 and 34 in the preferred implantation of this embodiment are in the range of 0. To 5 mm.

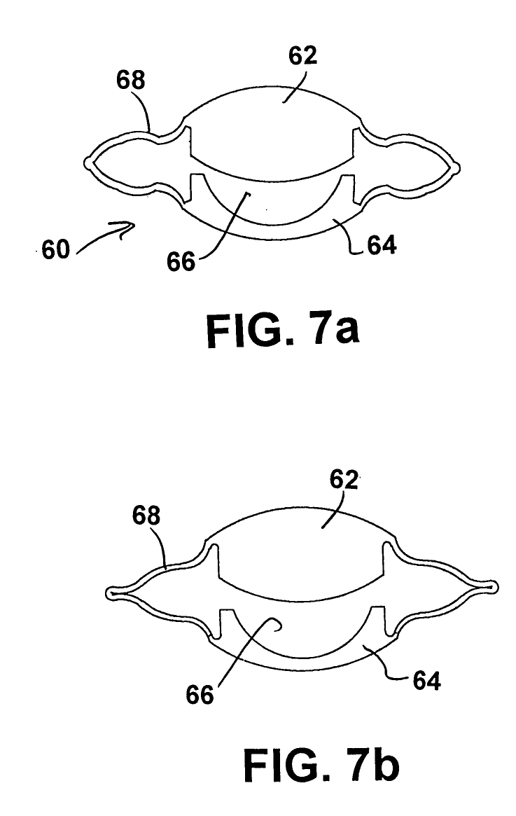

In another embodiment shown in FIGS. 7 and 8, a lens system 60 has a positive lens 62 and a negative lens 64 separated by a chamber 66. Chamber 66 may be filled with a gas (i.e., air) or a clear liquid (i.e., saline, water, et cetera). This embodiment is much like the embodiment of FIGS. 1-4, but ring 68 is fully enclosed to form a unitary chamber which permits internal chamber pressure to play a role in controlling separation between lenses 62 and 64. As shown in FIG. 7b, when the ring 68 is compressed (i.e., by the capsular bag) the increased pressure pushes lenses 62 and 64 further apart. Lenses 62 and 64 can be made of a high index material such as sapphire which has a refractive index of 1.77 which results in a smaller and thinner assembly.

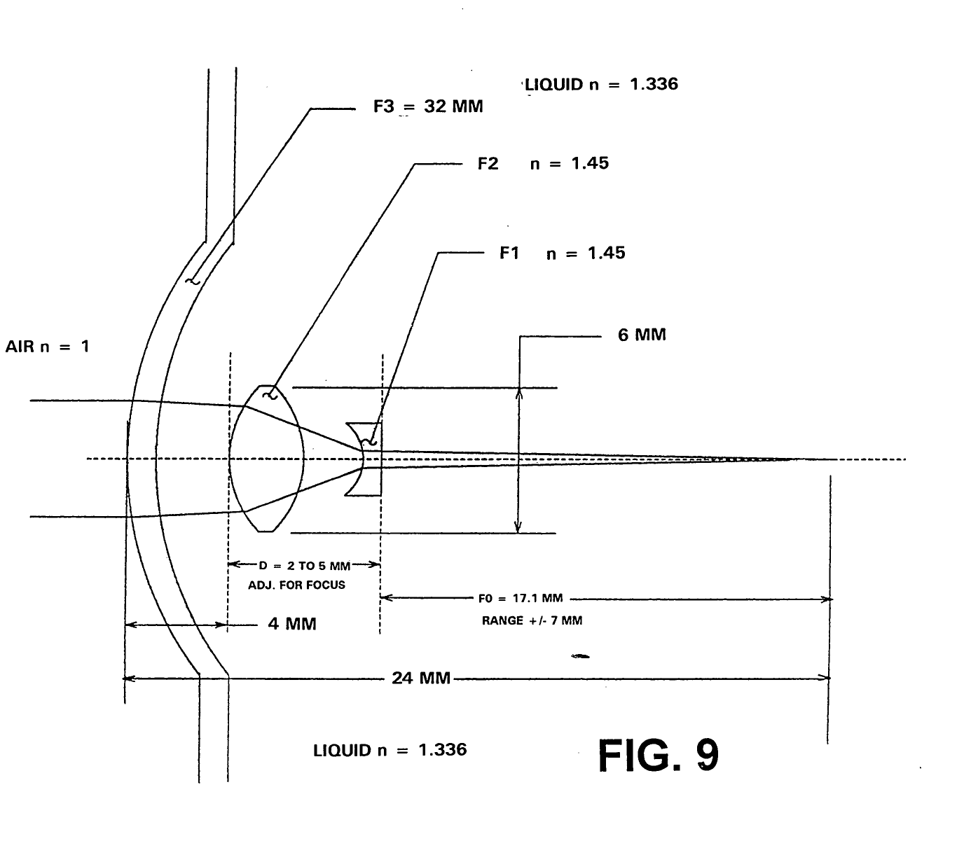

A key feature of the present invention is the high optical gain and large accommodation range of a lens system having spaced positive and negative lenses. As used herein, the term optical gain is the ratio of focal length change for the combined lenses to the change in distance between lenses to achieve that focal length change. FIG. 9 and Table 1 show what the gain and range are for the lens system of the present invention. FIG. 9 helps to define the terms used in Table 1. Table 1 specifies the optical gain and large range for a number of different positive and negative lenses. Thus for example, for F₁=−9 mm and F₂=+9 mm with D between 2.4 mm and 3.3 mm, the gain is between 14.7 and 7.7. This means that for a change in F₀ of 1 mm, the distance D has to change only about 0.07 to 0.13 mm. In another example, for F₁=−7 and F₂=+7 with D between 1.5 mm and 2.2 mm, the gain is between 23 and 10. This means that for a change in F₀ of 1 mm, the distance D has to change only about 0.04 mm to 0.1 mm.

In general terms, the combined focal length F₀ of the two lenses F₁ for the negative lens and F₂ for the positive lens is determined by the formula:

F₀=[F₂·(D−F₁)]/[D−(F₁+F₂)] (1)

where D=the distance between the two lenses. Moreover, the gain=ΔF₀/ΔD. From Table 1 it can be seen that for F₁ between −7 mm and −11 mm and F₂ between +7 and +12, the gain varies between about 6 and 23 for lens spacing between about 1.5 and 5 mm. Thus, for those values of F₁ and F₂, a change in the distance between the lenses will produce between 6 and 23 times that change in the focal length of the combined lens system. It can be seen in equation (1) that the reason for this large gain in a lens system having positive and negative lenses is that F_{1 and F}₂ are of opposite polarity thereby increasing the impact of D in the denominator on the value of F₀.

Thus, because the burden on the ciliary muscles to move the lenses relative to one another is reduced, the change in F₀ can be more readily accomplished over a wider range in the present invention then in the most relevant prior art implantable lens systems. Moreover, the unique structure of each of the disclosed embodiments, assures that the respective lenses always remain axially aligned with one another to assure excellent optical performance over a period of decades. Thus, for example in FIGS. 5a and 5b, it is seen that the lenses 32 and 34 remain axially centered and aligned along centerline 35 whether separated or not. Moreover, because of the unique scissor-like operation, even unbalanced activation forces will not displace either lens from that centerline.

TABLE 1

| F1 | F2 | D | F0 | GAIN | F1 | F2 | D | F0 | GAIN | F1 | F2 | D | F0 | GAIN | F1 | F2 | D | F0 | GAIN |
|---|---|---|---|---|---|---|---|---|---|---|---|---|---|---|---|---|---|---|---|
| −7.0 | 7.0 | 1.50 | 25.7 | 23.3 | −7.0 | 8.0 | 2.50 | 25.7 | 23.3 | −7.0 | 9.0 | 3.50 | 25.7 | 23.3 | −7.0 | 10.0 | 4.50 | 25.7 | 23.3 |
| −7.0 | 7.0 | 1.60 | 23.6 | 20.4 | −7.0 | 8.0 | 2.60 | 23.6 | 20.4 | −7.0 | 9.0 | 3.60 | 23.6 | 20.4 | −7.0 | 10.0 | 4.60 | 23.6 | 20.4 |
| −7.0 | 7.0 | 1.70 | 21.8 | 18.0 | −7.0 | 8.0 | 2.70 | 21.8 | 18.0 | −7.0 | 9.0 | 3.70 | 21.8 | 18.0 | −7.0 | 10.0 | 4.70 | 21.8 | 18.0 |
| −7.0 | 7.0 | 1.80 | 20.2 | 16.0 | −7.0 | 8.0 | 2.80 | 20.2 | 16.0 | −7.0 | 9.0 | 3.80 | 20.2 | 16.0 | −7.0 | 10.0 | 4.80 | 20.2 | 16.0 |
| −7.0 | 7.0 | 1.90 | 18.8 | 14.3 | −7.0 | 8.0 | 2.90 | 18.8 | 14.3 | −7.0 | 9.0 | 3.90 | 18.8 | 14.3 | −7.0 | 10.0 | 4.90 | 18.8 | 14.3 |
| −7.0 | 7.0 | 2.00 | 17.5 | 12.9 | −7.0 | 8.0 | 3.00 | 17.5 | 12.9 | −7.0 | 9.0 | 4.00 | 17.5 | 12.9 | −7.0 | 10.0 | 5.00 | 17.5 | 12.9 |
| −7.0 | 7.0 | 2.10 | 16.3 | 11.7 | −7.0 | 8.0 | 3.10 | 16.3 | 11.7 | −7.0 | 9.0 | 4.10 | 16.3 | 11.7 | −7.0 | 10.0 | 5.10 | 16.3 | 11.7 |
| −7.0 | 7.0 | 2.20 | 15.3 | 10.6 | −7.0 | 8.0 | 3.20 | 15.3 | 10.6 | −7.0 | 9.0 | 4.20 | 15.3 | 10.6 | −7.0 | 10.0 | 5.20 | 15.3 | 10.6 |
| −9.0 | 9.0 | 2.40 | 24.8 | 14.7 | −9.0 | 10.0 | 3.40 | 24.8 | 14.7 | −9.0 | 11.0 | 4.40 | 24.8 | 14.7 | −9.0 | 12.0 | 5.40 | 24.8 | 14.7 |
| −9.0 | 9.0 | 2.50 | 23.4 | 13.5 | −9.0 | 10.0 | 3.50 | 23.4 | 13.5 | −9.0 | 11.0 | 4.50 | 23.4 | 13.5 | −9.0 | 12.0 | 5.50 | 23.4 | 13.5 |
| −9.0 | 9.0 | 2.60 | 22.2 | 12.5 | −9.0 | 10.0 | 3.60 | 22.2 | 12.5 | −9.0 | 11.0 | 4.60 | 22.2 | 12.5 | −9.0 | 12.0 | 5.60 | 22.2 | 12.5 |
| −9.0 | 9.0 | 2.70 | 21.0 | 11.5 | −9.0 | 10.0 | 3.70 | 21.0 | 11.5 | −9.0 | 11.0 | 4.70 | 21.0 | 11.5 | −9.0 | 12.0 | 5.70 | 21.0 | 11.5 |
| −9.0 | 9.0 | 2.80 | 19.9 | 10.7 | −9.0 | 10.0 | 3.80 | 19.9 | 10.7 | −9.0 | 11.0 | 4.80 | 19.9 | 10.7 | −9.0 | 12.0 | 5.80 | 19.9 | 10.7 |
| −9.0 | 9.0 | 2.90 | 18.9 | 10.0 | −9.0 | 10.0 | 3.90 | 18.9 | 10.0 | −9.0 | 11.0 | 4.90 | 18.9 | 10.0 | −9.0 | 12.0 | 5.90 | 18.9 | 10.0 |
| −9.0 | 9.0 | 3.00 | 18.0 | 9.3 | −9.0 | 10.0 | 4.00 | 18.0 | 9.3 | −9.0 | 11.0 | 5.00 | 18.0 | 9.3 | −9.0 | 12.0 | 6.00 | 18.0 | 9.3 |
| −9.0 | 9.0 | 3.10 | 17.1 | 8.7 | −9.0 | 10.0 | 4.10 | 17.1 | 8.7 | −9.0 | 11.0 | 5.10 | 17.1 | 8.7 | −9.0 | 12.0 | 6.10 | 17.1 | 8.7 |
| −9.0 | 9.0 | 3.20 | 16.3 | 8.2 | −9.0 | 10.0 | 4.20 | 16.3 | 8.2 | −9.0 | 11.0 | 5.20 | 16.3 | 8.2 | −9.0 | 12.0 | 6.20 | 16.3 | 8.2 |
| −9.0 | 9.0 | 3.30 | 15.5 | 7.7 | −9.0 | 10.0 | 4.30 | 15.5 | 7.7 | −9.0 | 11.0 | 5.30 | 15.5 | 7.7 | −9.0 | 12.0 | 6.30 | 15.5 | 7.7 |
| −9.0 | 9.0 | 3.40 | 14.8 | 7.2 | −9.0 | 10.0 | 4.40 | 14.8 | 7.2 | −9.0 | 11.0 | 5.40 | 14.8 | 7.2 | −9.0 | 12.0 | 6.40 | 14.8 | 7.2 |
| −10.0 | 9.0 | 1.90 | 24.5 | 12.3 | −10.0 | 10.0 | 2.90 | 24.5 | 12.3 | −10.0 | 11.0 | 3.90 | 24.5 | 12.3 | −10.0 | 12.0 | 4.90 | 24.5 | 12.3 |
| −10.0 | 9.0 | 2.00 | 23.3 | 11.5 | −10.0 | 10.0 | 3.00 | 23.3 | 11.5 | −10.0 | 11.0 | 4.00 | 23.3 | 11.5 | −10.0 | 12.0 | 5.00 | 23.3 | 11.5 |
| −10.0 | 9.0 | 2.10 | 22.3 | 10.8 | −10.0 | 10.0 | 3.10 | 22.3 | 10.6 | −10.0 | 11.0 | 4.10 | 22.3 | 10.8 | −10.0 | 12.0 | 5.10 | 22.3 | 10.8 |
| −10.0 | 9.0 | 2.20 | 21.3 | 10.1 | −10.0 | 10.0 | 3.20 | 21.3 | 10.1 | −10.0 | 11.0 | 4.20 | 21.3 | 10.1 | −10.0 | 12.0 | 5.20 | 21.3 | 10.1 |
| −10.0 | 9.0 | 2.30 | 20.3 | 9.5 | −10.0 | 10.0 | 3.30 | 20.3 | 9.5 | −10.0 | 11.0 | 4.30 | 20.3 | 9.5 | −10.0 | 12.0 | 5.30 | 20.3 | 9.5 |
| −10.0 | 9.0 | 2.40 | 19.4 | 8.9 | −10.0 | 10.0 | 3.40 | 19.4 | 8.9 | −10.0 | 11.0 | 4.40 | 19.4 | 8.9 | −10.0 | 12.0 | 5.40 | 19.4 | 8.9 |
| −10.0 | 9.0 | 2.50 | 18.6 | 8.4 | −10.0 | 10.0 | 3.50 | 18.6 | 8.4 | −10.0 | 11.0 | 4.50 | 18.6 | 8.4 | −10.0 | 12.0 | 5.50 | 18.6 | 8.4 |
| −10.0 | 9.0 | 2.60 | 17.8 | 7.9 | −10.0 | 10.0 | 3.60 | 17.8 | 7.9 | −10.0 | 11.0 | 4.60 | 17.8 | 7.9 | −10.0 | 12.0 | 5.60 | 17.8 | 7.9 |
| −10.0 | 9.0 | 2.70 | 17.0 | 7.6 | −10.0 | 10.0 | 3.70 | 17.0 | 7.5 | −10.0 | 11.0 | 4.70 | 17.0 | 7.5 | −10.0 | 12.0 | 5.70 | 17.0 | 7.5 |
| −10.0 | 9.0 | 2.80 | 16.3 | 7.1 | −10.0 | 10.0 | 3.80 | 16.3 | 7.1 | −10.0 | 11.0 | 4.80 | 16.3 | 7.1 | −10.0 | 12.0 | 5.80 | 16.3 | 7.1 |
| −10.0 | 9.0 | 2.90 | 15.6 | 6.7 | −10.0 | 10.0 | 3.90 | 15.6 | 6.7 | −10.0 | 11.0 | 4.90 | 15.6 | 6.7 | −10.0 | 12.0 | 5.90 | 15.6 | 6.7 |
| −10.0 | 9.0 | 3.00 | 15.0 | 6.4 | −10.0 | 10.0 | 4.00 | 15.0 | 6.4 | −10.0 | 11.0 | 5.00 | 15.0 | 6.4 | −10.0 | 12.0 | 6.00 | 15.0 | 6.4 |
| −11.0 | 9.0 | 1.40 | 24.6 | 10.8 | −11.0 | 10.0 | 2.40 | 24.6 | 10.8 | −11.0 | 11.0 | 3.40 | 24.6 | 10.8 | −11.0 | 12.0 | 4.40 | 24.6 | 10.8 |
| −11.0 | 9.0 | 1.50 | 23.6 | 10.2 | −11.0 | 10.0 | 2.50 | 23.6 | 10.2 | −11.0 | 11.0 | 3.50 | 23.6 | 10.2 | −11.0 | 12.0 | 4.50 | 23.6 | 10.2 |
| −11.0 | 9.0 | 1.60 | 22.6 | 9.6 | −11.0 | 10.0 | 2.60 | 22.6 | 9.6 | −11.0 | 11.0 | 3.60 | 22.6 | 9.6 | −11.0 | 12.0 | 4.60 | 22.6 | 9.6 |
| −11.0 | 9.0 | 1.70 | 21.7 | 9.1 | −11.0 | 10.0 | 2.70 | 21.7 | 9.1 | −11.0 | 11.0 | 3.70 | 21.7 | 9.1 | −11.0 | 12.0 | 4.70 | 21.7 | 9.1 |
| −11.0 | 9.0 | 1.80 | 20.8 | 8.6 | −11.0 | 10.0 | 2.80 | 20.8 | 8.6 | −11.0 | 11.0 | 3.80 | 20.8 | 8.6 | −11.0 | 12.0 | 4.80 | 20.8 | 8.6 |
| −11.0 | 9.0 | 1.90 | 20.0 | 8.2 | −11.0 | 10.0 | 2.90 | 20.0 | 8.2 | −11.0 | 11.0 | 3.90 | 20.0 | 8.2 | −11.0 | 12.0 | 4.90 | 20.0 | 8.2 |
| −11.0 | 9.0 | 2.00 | 19.3 | 7.8 | −11.0 | 10.0 | 3.00 | 19.3 | 7.8 | −11.0 | 11.0 | 4.00 | 19.3 | 7.8 | −11.0 | 12.0 | 5.00 | 19.3 | 7.8 |
| −11.0 | 9.0 | 2.10 | 18.5 | 7.4 | −11.0 | 10.0 | 3.10 | 18.5 | 7.4 | −11.0 | 11.0 | 4.10 | 18.5 | 7.4 | −11.0 | 12.0 | 5.10 | 18.5 | 7.4 |
| −11.0 | 9.0 | 2.20 | 17.8 | 7.0 | −11.0 | 10.0 | 3.20 | 17.8 | 7.0 | −11.0 | 11.0 | 4.20 | 17.8 | 7.0 | −11.0 | 12.0 | 5.20 | 17.8 | 7.0 |
| −11.0 | 9.0 | 2.30 | 17.1 | 6.7 | −11.0 | 10.0 | 3.30 | 17.1 | 6.7 | −11.0 | 11.0 | 4.30 | 17.1 | 6.7 | −11.0 | 12.0 | 5.30 | 17.1 | 6.7 |
| −11.0 | 9.0 | 2.40 | 16.5 | 6.4 | −11.0 | 10.0 | 3.40 | 16.5 | 6.4 | −11.0 | 11.0 | 4.40 | 16.5 | 6.4 | −11.0 | 12.0 | 5.40 | 16.5 | 6.4 |
| −11.0 | 9.0 | 2.50 | 15.9 | 6.1 | −11.0 | 10.0 | 3.50 | 15.9 | 6.1 | −11.0 | 11.0 | 4.50 | 15.9 | 6.1 | −11.0 | 12.0 | 5.50 | 15.9 | 6.1 |
| −11.0 | 9.0 | 2.60 | 15.3 | 5.8 | −11.0 | 10.0 | 3.60 | 15.3 | 5.8 | −11.0 | 11.0 | 4.60 | 15.3 | 5.8 | −11.0 | 12.0 | 5.60 | 15.3 | 5.8 |
| −11.0 | 9.0 | 2.70 | 14.7 | 5.6 | −11.0 | 10.0 | 3.70 | 14.7 | 5.6 | −11.0 | 11.0 | 4.70 | 14.7 | 5.6 | −11.0 | 12.0 | 5.70 | 14.7 | 5.6 |

Having thus disclosed preferred embodiments of the invention, it being understood that various alternative embodiments are contemplated and that the protection afforded hereby is not limited by the disclosed embodiments but only by the appended claims and their equivalents, what is claimed is:

1. An intraocular replacement lens system for implant into a capsular bag of an eye, said lens system comprising:
a first lens having a first edge;
a second lens having a second edge; and
at least one fulcrum connecting said first and second edge to place said first lens and second lens at a predetermined distance from each other and to place said lenses along a common axis and to constrain movement of said lenses along the common optical axis.

2. The intraocular replacement lens system of claim 1 further comprising:
a first haptic member connected to said at least one function at said first edge; and
a second haptic member connected to said at least one fulcrum at said second edge, the connection of said first and second haptic members at said at least one fulcrum providing a scissor-type mechanism having a lever effect on said first and second lenses to produce movement of said lenses along the common optical axis when said haptic members are moved toward or away from each other, said distance between said lenses determining the focal length of said lens system.

3. The intraocular replacement lens system of claim 2 wherein direction of movement of said first and second haptic members is inversely proportional to the direction of movement of said first and second lens such that when said haptic members are moved toward each other said lenses move away from each other and vice versa.

4. The intraocular replacement lens system of claim 2 wherein said at least one fulcrum is formed from material selected from the group comprising plastic hinges, annular pivots and narrow flexible joints.

5. The intraocular replacement lens system of claim 4 wherein said lens system is a one-piece assembly.

6. The intraocular replacement lens system of claim 2 wherein said first lens is a positive lens and said second lens is a negative lens.

7. The intraocular replacement lens system of claim 6 wherein said first and second lenses are formed from material selected from the group comprising a high index material, sapphire, plastic, acrylic and acrylic polymers.

8. The intraocular replacement lens system of claim 6 wherein when said lens system is implanted into said capsular bag of said eye, said first and second haptic members extend toward an inner edge of said capsular bag which is adjacent to ciliary muscles and ciliary fibers of said eye.

9. The intraocular replacement lens system of claim 8 wherein when said lens system is implanted into said capsular bag of said eye, said positive lens is oriented closer to the iris of said eye while said negative lens is oriented away from said iris.

10. The intraocular replacement lens system of claim 9 wherein when said ciliary muscles contract, said capsular bag is stretched thereby moving said haptic members toward each other causing said lenses to move away from each other shortening the focal length of said lens system to permit focusing of object at near distances.

11. The intraocular replacement lens system of claim 10 wherein when said ciliary muscles relax, said capsular bag relaxes thereby moving said haptic members away from each other causing said lenses to move toward each other increasing the focal length of said lens system to permit focusing of objects from far distances.

12. The intraocular replacement lens system of claim 11 wherein said scissor-type mechanism assures continuous alignment of said lenses along said common optical axis regardless of the application of unbalanced forces at said first and second haptic member.

13. The intraocular replacement lens system of claim 12 wherein combination of said positive and negative lenses of said intraocular lens system results in a high optical gain and a large focusing range of said lens system.

14. The intraocular replacement lens system of claim 13 wherein said first and second lenses are positioned so that their respective closest surface points are at a predetermined distance from each other such that movement of said lenses along said common optical axis maintains a proper focal plane on a retina of said eye.

15. The intraocular replacement lens system of claim 14 wherein said positive lens has a focal length between +5 mm to +12 mm and said negative lens has a focal length between −5 mm to −12 mm.

16. The intraocular replacement lens system of claim 15 wherein said predetermined distance between said first and second lens is between 0 mm to 5 mm.

17. A method for making an intraocular replacement lens system for implant into a capsular bag of an eye, said method for making comprising the steps of:
providing a first lens having a first edge;
providing a second lens having a second edge;
providing at least one fulcrum; and
connecting said at least one fulcrum to said first and second edges to place said first lens and second lens at a predetermined distance from each other and place said lenses along a common optical axis and constrain movement of said lenses along said common optical axis.

18. The method of claim 17 further comprising the steps of:
providing a first haptic member;
connecting said first haptic member to said at least one fulcrum at said first edge;
providing a second haptic member; and
connecting said second haptic member to said at least one fulcrum at said second edge, the connection of said first and second haptic members at said fulcrum results in a scissor-type mechanism providing a lever effect on said first and second lenses to produce movement of said lenses along said common optical axis when said haptic members are moved toward or away from each other to thereby establish gain, said distance between said lenses determines a focal length of said lens system.

19. An intraocular replacement lens system for implant into a capsular bag of an eye, said lens system comprising:
a first lens having a first edge;
a first flex interface connected to said first lens at said first edge;
a second flex interface connected to said first lens at said first edge, said second flex interface located opposite said first flex interface;
a second lens having a second edge;
a third flex interface connected to said second lens at said second edge;
a fourth flex interface connected to said second lens at said second edge, said fourth flex interface located opposite said third flex interface;
a first haptic member attached to said first and second flex interface at a bend point at each of said first and second flex interface;
a second haptic member attached to said first and second flex interface at a bend point at each of said first and second flex interface, said second haptic member located opposite said first haptic member;
a third haptic member attached to said third and fourth flex interfaces at a bend point at each of said third and fourth flex interface;
a fourth haptic member attached to said third and fourth flex interface at a bend point at each of said third and fourth flex interface, said fourth haptic member located opposite said third haptic member;
a first fulcrum connecting said first and third haptic members; and
a second fulcrum connecting said second and fourth haptic members to place said first lens and second lens at a predetermined distance from each other and place said lenses along a common optical axis and constrain movement of said lenses along said common optical axis.

20. The intraocular replacement lens system according to claim 19 wherein said first, second, third and fourth haptic members are oriented parallel to said first and second edges.

21. The intraocular replacement lens system according to claim 20 wherein when said first and third haptic members are moved toward each other, said first and second lens move away from each other.

22. The intraocular replacement lens system according to claim 21 wherein said second and fourth haptic members are moved toward each other, said first and second lens move away from each other.

23. A lens system for implant into the capsular bag of a human eye in place of the natural crystalline lens, the lens system comprising:
a pair of interconnected hinged lenses spaced from one another along a common optical axis, the relative spacing between the lenses being controllable along said axis by the human eye after implanting therein, said spacing determining the focal length of said lens system, one of said lenses having a focal length in the range of 5 mm to 12 mm and the other of said lenses having a focal length in the range of −5 mm to −12 mm whereby a change in said spacing produces a change in said focal length of said lens system which is between 6 and at least 23 times said spacing change.
